(19) United States
(12) Patent Application Publication
(10) Pub. No.: US 2003/0162222 A1
(43) Pub. Date: Aug. 28, 2003

(54) Method for selecting enzyme inhibitors

(76) Inventors: Markus Warmuth, Munchen (DE); Ruth Mathes, Munchen (DE); Michael Hallek, Schondorf (DE)

Correspondence Address: Thomas A Miller, Gregory J Hartwig, Michael Best & Friedrich, 100 East Wisconsin Avenue, Milwaukee, WI, 53202, US

(21) Appl. No.: 10/276,633

(22) PCT Filed: May 17, 2001
(86) PCT No.: PCT/EP01/05661

(30) Foreign Application Priority Data
May 17, 2000 (DE) 100 24 174.3

(51) Int. Cl. G01N 33/53; C12N 9/12; C12N 9/16; C12N 9/64; C12P 21/02

(52) U.S. Cl. 435/7.1; 435/69.1; 435/194; 435/196; 435/226

(57) ABSTRACT

The invention relates to a method of selecting inhibitors for enzymes and use of the selected inhibitor as a therapeutic and/or prophylactic agent, a mutant which can be used in the method and a method of testing an inhibitor for its specificity in a modelling system.

The invention relates to a method of selecting inhibitors for enzymes and use of the selected inhibitor as a therapeutic and/or prophylactic agent, a mutant which may be used in the method and a method of testing an inhibitor for its specificity in a modelling system

In screening for new lead structures for enzyme inhibitors, e.g. kinase inhibitors, a substance library is traditionally tested against a number of other enzymes such as kinases. The lead structures which specifically inhibit the enzyme tested but none of the other enzymes are then selected.

A method of this type is described in Hanke et al., The Journal of Biological Chemistry, 1996, 271, pp. 695-701. Two tyrosine kinase inhibitors with high selectivity for Src kinases compared to a series of other cellular protein kinases such as the ZAP70, Jak2 or PKA kinases and EGF receptor kinase have been found by this method. These inhibitors are the following pyrazol pyrimidine derivatives PP1 and PP2.

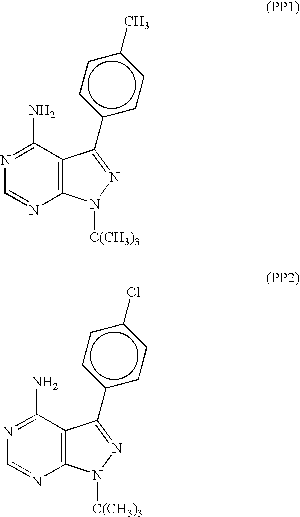

It has also been discovered by known screening methods that inhibitor STI571 (formerly CGP57148) specifically inhibits the tyrosine kinase c-Abl (Buchdunger et al, Cancer Research, 1996, 56, S. 100-104).

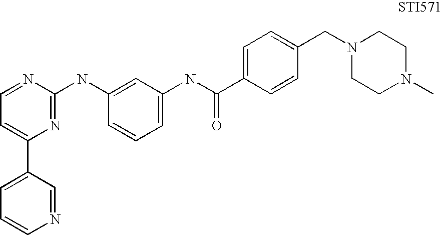

This type of screening is difficult to carry out, time consuming and expensive, especially owing to the time taken to establish the necessary assay arrays. Every enzyme to be tested, e.g. a kinase, has to be expressed and purified. The optimum reaction conditions then have to be found for each enzyme, e.g. each kinase. Development costs and the time spent on development are enormous, according to the size of the assay arrays. And it is not possible to test the biological activity of the inhibitors found, as to whether they are specific to the desired inhibition of a certain enzyme

The problem of the invention is therefore to provide a method of selecting inhibitors which is simple, cost-effective and not time-intensive.

The solution to the problem is a method of selecting inhibitors, comprising the steps of:

a) finding a binding site in a wt (wild type) enzyme which is inhibitor-specific but not substrate-specific,

b) replacing at least one amino acid at the binding site of the wt enzyme which is found to be inhibitor-specific with a different amino acid, whereby a mutant of the wt enzyme is obtained,

c) testing the mutants obtained at step b) for enzyme activity and selecting the active mutants,

d) testing at least one substance with the wt enzyme and the mutant selected at step c), and

e) selecting the substance as inhibitor if it inhibits the wt enzyme but not the mutant selected at step c).

Another problem on which the invention is based is to provide mutants for use in the method of the invention.

The solution to that problem is a mutant of a wt enzyme which can be obtained by carrying out steps a) and b) of the method of the invention.

A further problem underlying the invention is to provide a simple, safe method of testing an inhibitor for biological effects specific to the interaction between the inhibitor and the inhibitor-specific binding site.

The solution to the problem is a method of testing an inhibitor selected by the method of the invention for biological effects specific to the interaction between the inhibitor and the inhibitor-specific binding site, comprising the steps of:

1) incubating a modelling system in which the wt enzyme is expressed, with an inhibitor,

2) establishing the effects thereby obtained

3) incubating a modelling system, in which the inhibitor-resistant mutant according to the invention is expressed, with the inhibitor,

4) establishing the effects thereby obtained

5) comparing the effects found at step 2) and step 4), and

6) selecting the effects found at step 2) but not step 4), as being specific to the interaction between the inhibitor and the inhibitor-specific binding site.

The invention will be explained below with reference to the figures inter alia.

In these:

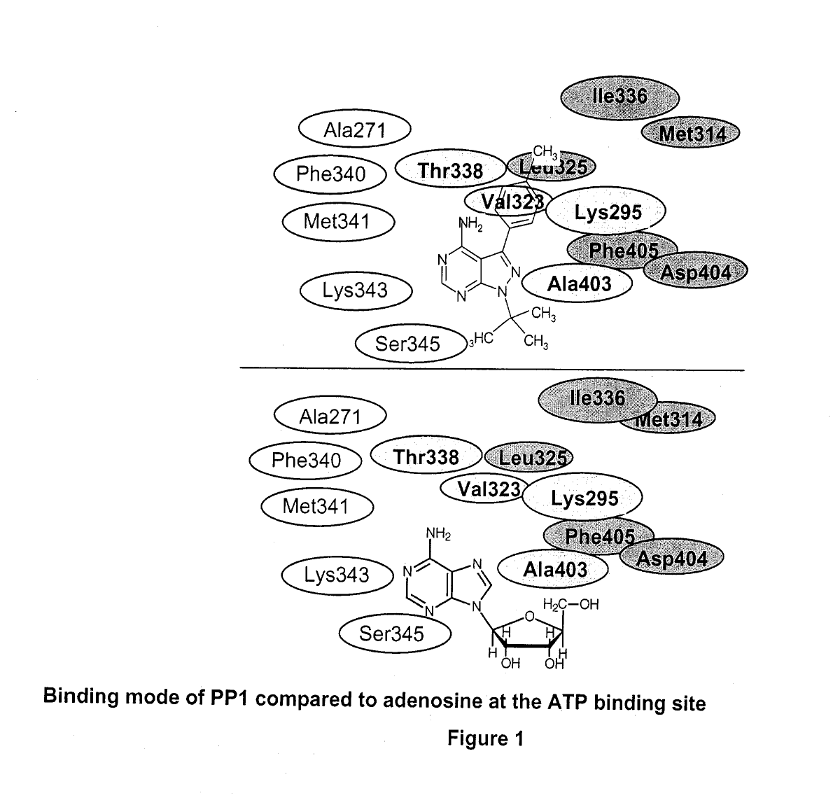

FIG. 1 shows the binding mode of PP1 compared to adenosine at the binding site of Src kinase Hck;

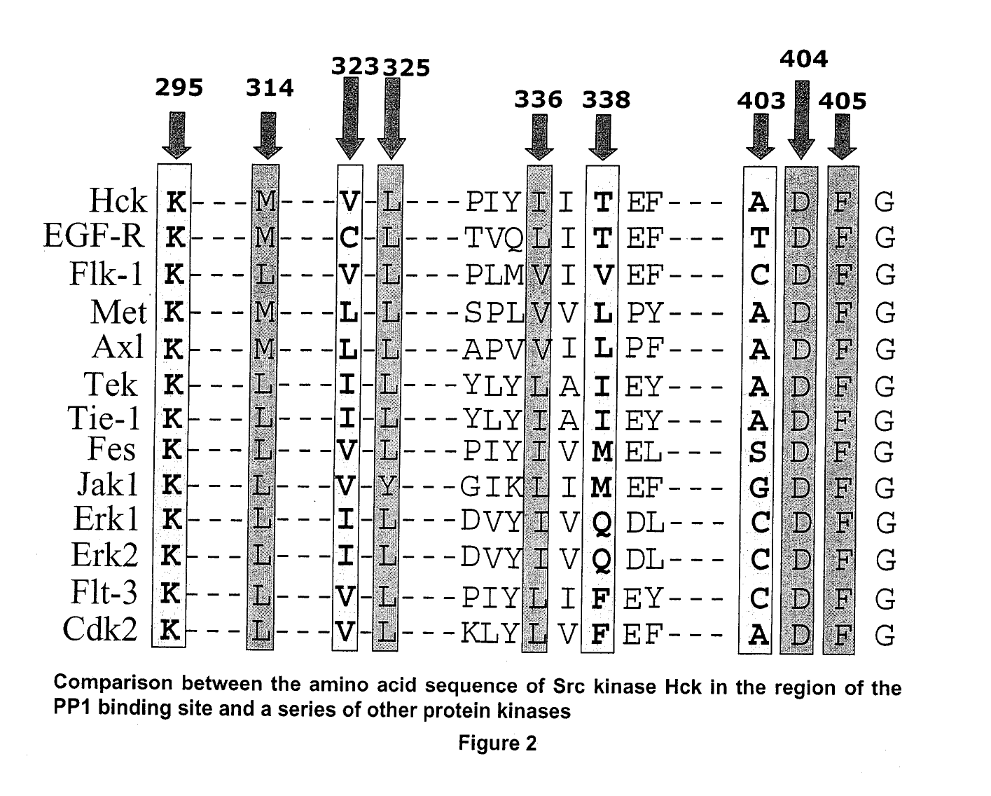

FIG. 2 is a comparison between the amino acid sequence of Src kinase Hck in the region of the PP1 binding site and a series of other protein kinases;

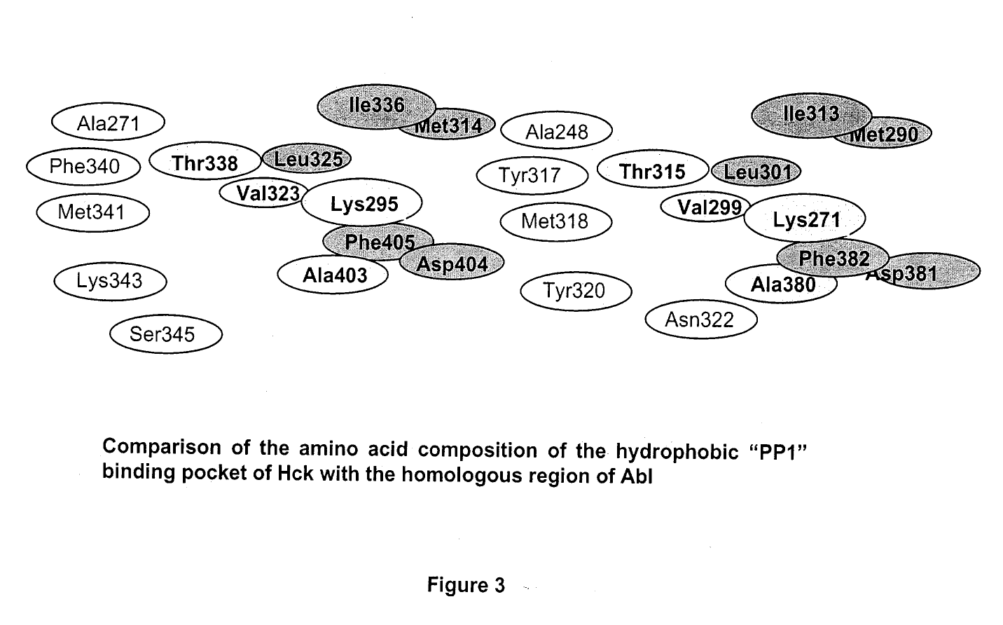

FIG. 3 is a comparison between the amino acid composition of the hydrphobic “PP1” binding pocket of Src kinase Hck and the homologous region of protein kinase Abl;

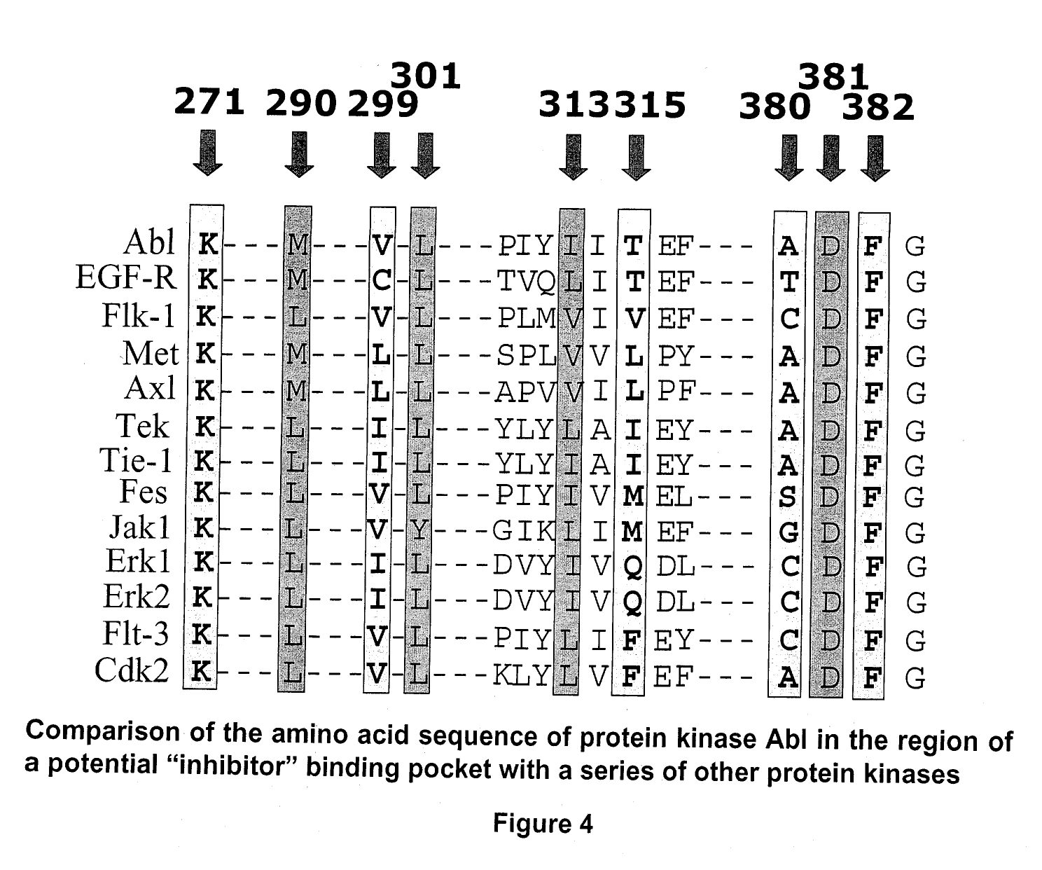

FIG. 4 is a comparison between the amino acid sequence of protein kinase Abl in the region of a potential “inhibitor” binding pocket and a series of other protein kinases;

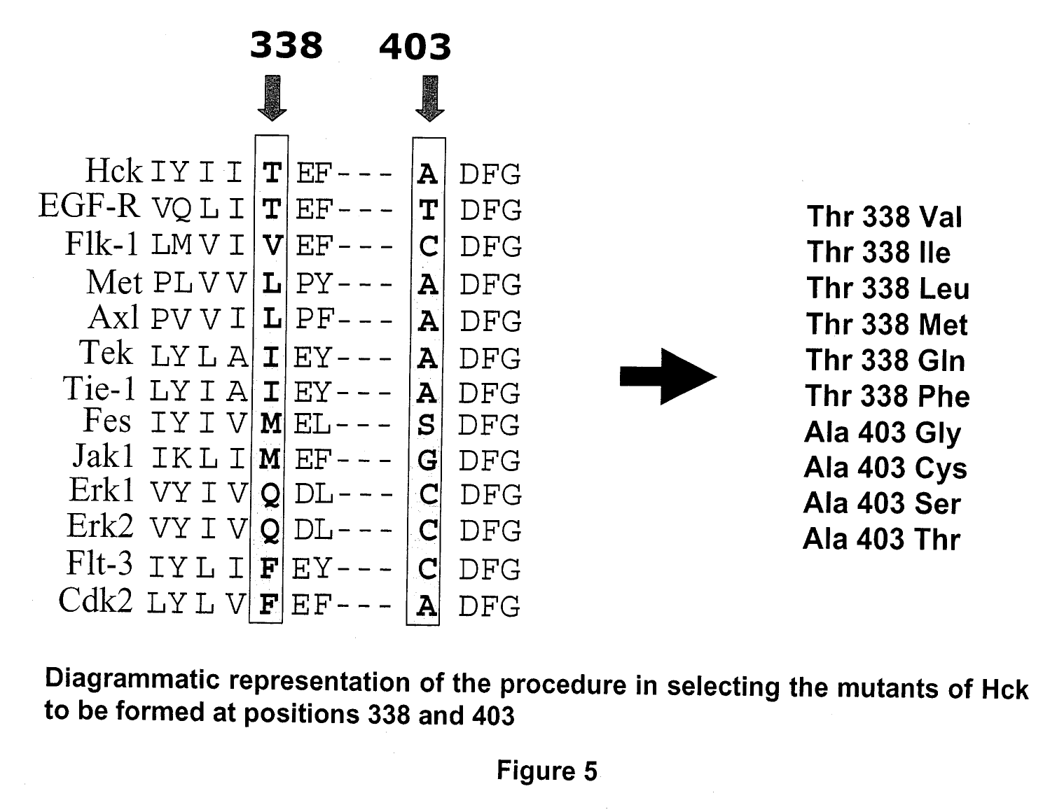

FIG. 5 is a diagrammatic representation of the procedure for selecting the mutants of Src kinase Hck to be formed at positions 338 and 403;

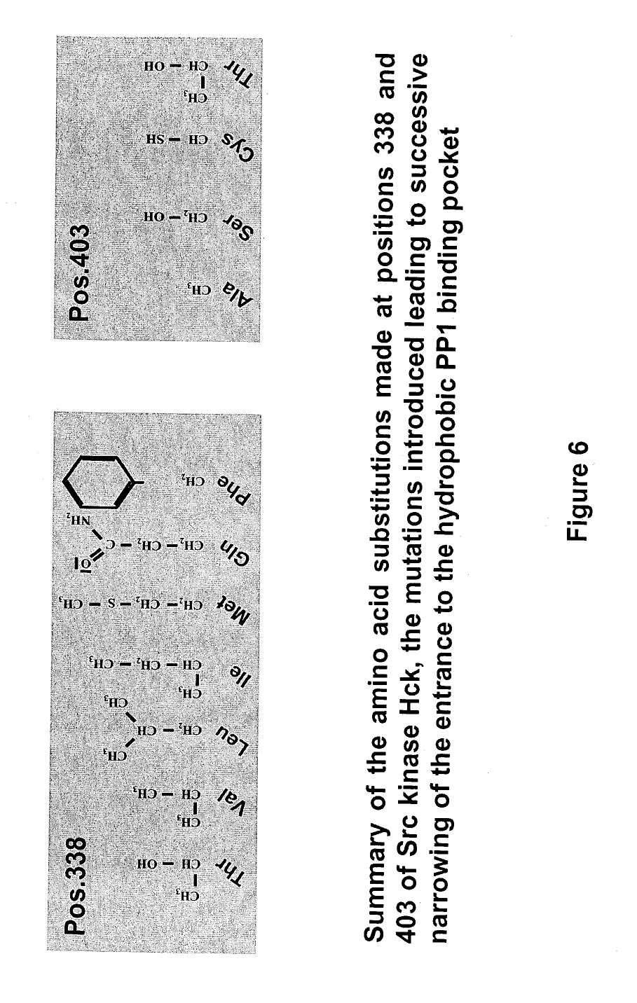

FIG. 6 summarises the amino acid substitutions made at positions 338 and 403 of Src kinase Hck, the mutations introduced leading to successive narrowing of the entrance to the hydrophobic PP1 binding pocket;

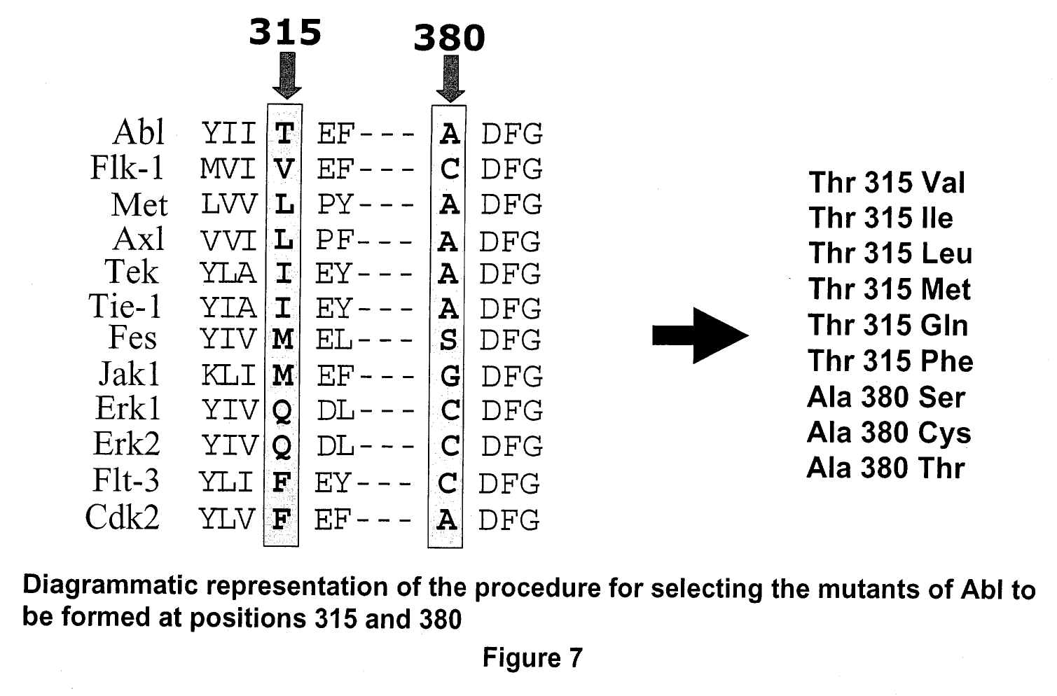

FIG. 7 is a diagrammatic representation of the procedure for selecting the mutants of protein kinase Abl to be made at positions 315 and 380;

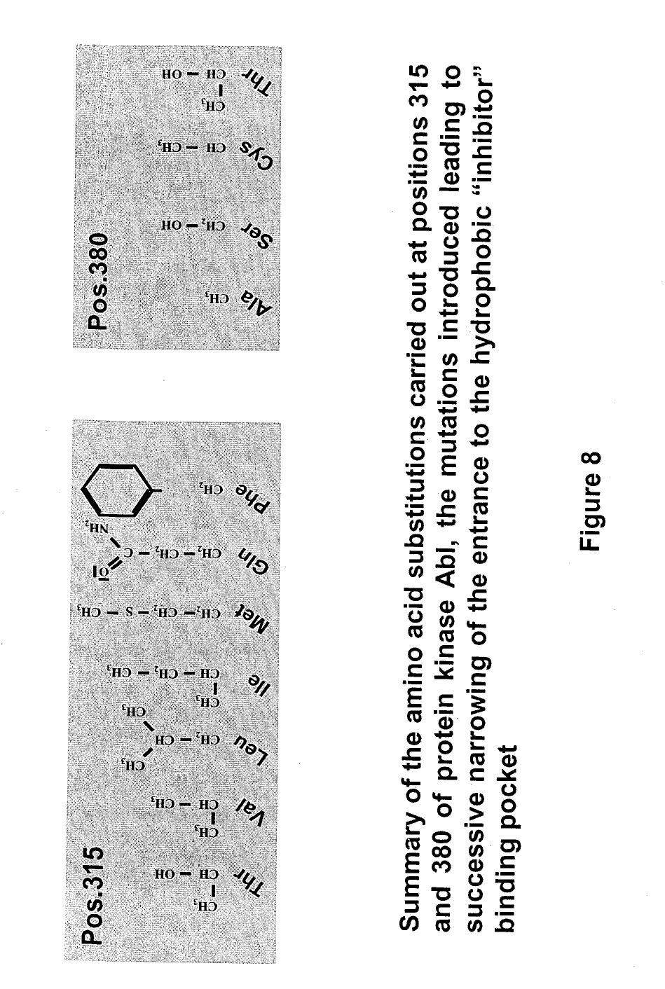

FIG. 8 summarises the amino acid substitutions carried out at positions 315 and 380 of protein kinase Abl, the mutations introduced leading to successive narrowing of the entrance to the hydrophobic “inhibitor” binding pocket;

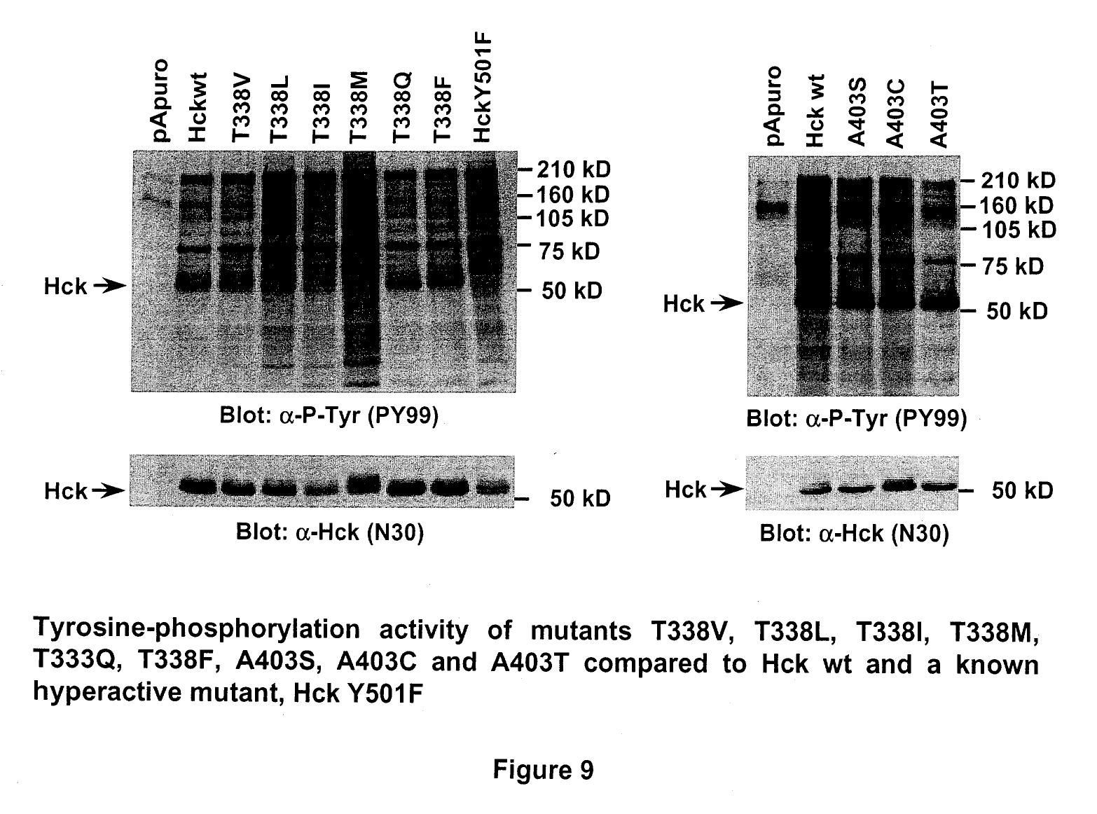

FIG. 9 shows the tyrosine-phosphorylation activity of mutants T338V, T338L, T338I, T338M, T338Q, T338F, A403S, A403C and A403T compared to wt Src kinase Hck and a known hyperactive mutant, Hck Y501 F;

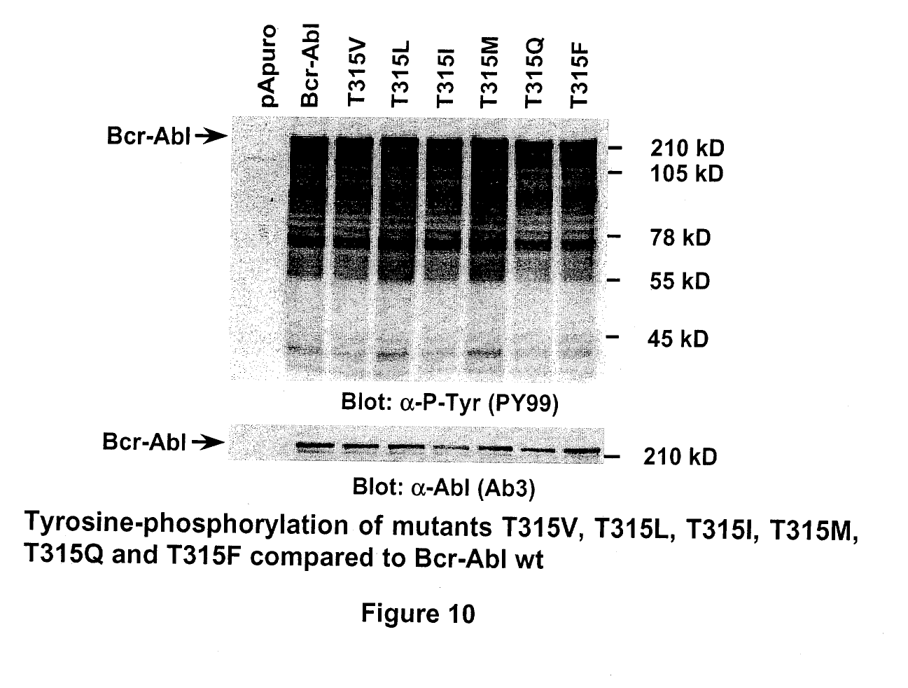

FIG. 10 shows the tyrosine-phosphorylation activity of mutants T315V, T315L, T315I, T315M, T315Q and T315F compared to wt Bcr-Abl;

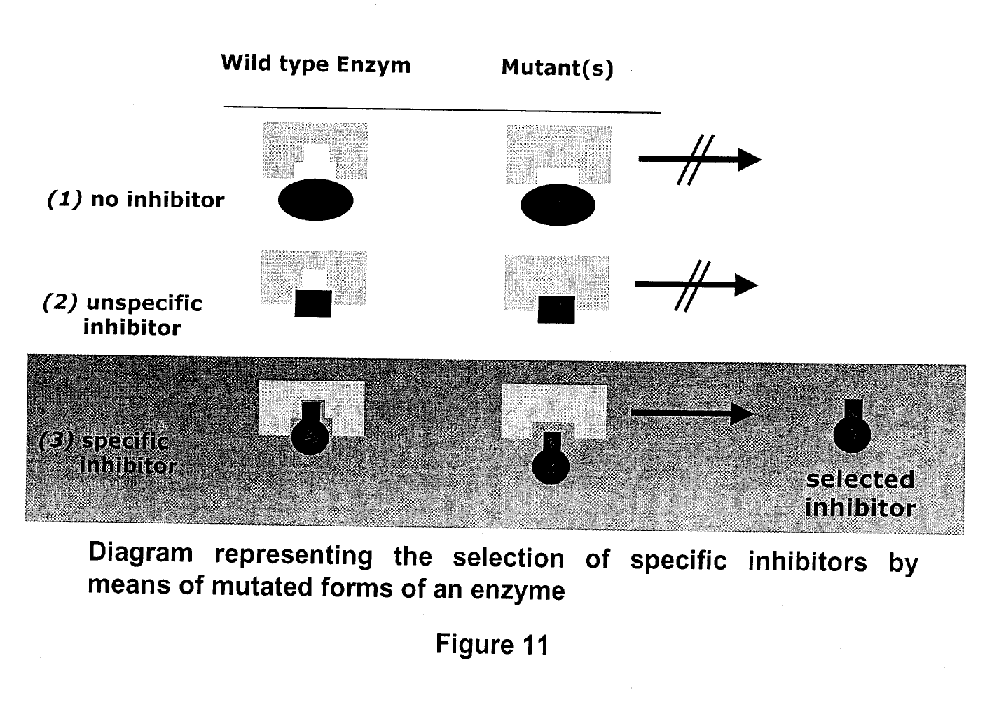

FIG. 11 is a diagram representing the selection of specific inhibitors by means of mutated forms of an enzyme;

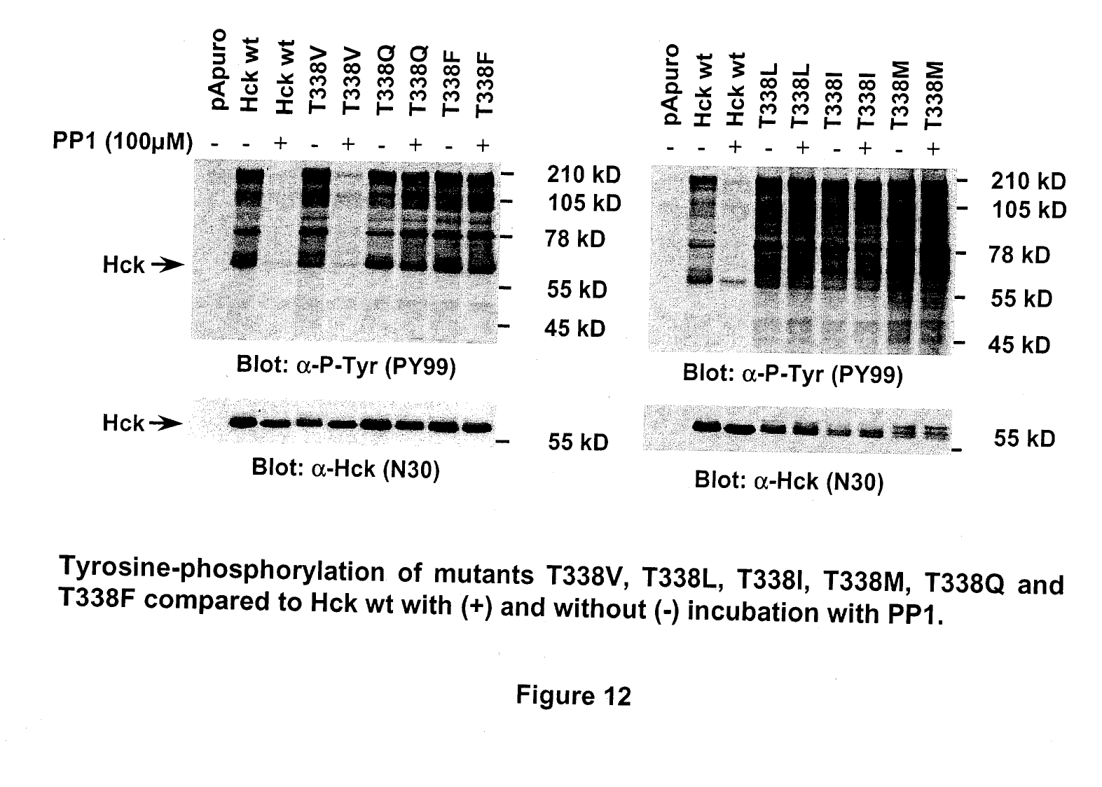

FIG. 12 shows the tyrosine-phosphorylation activity of mutants T338V, T338L, T338I, T338M, T338Q and T338F compared to wt Src kinase Hck with (+) and without (−) incubation with PP1;

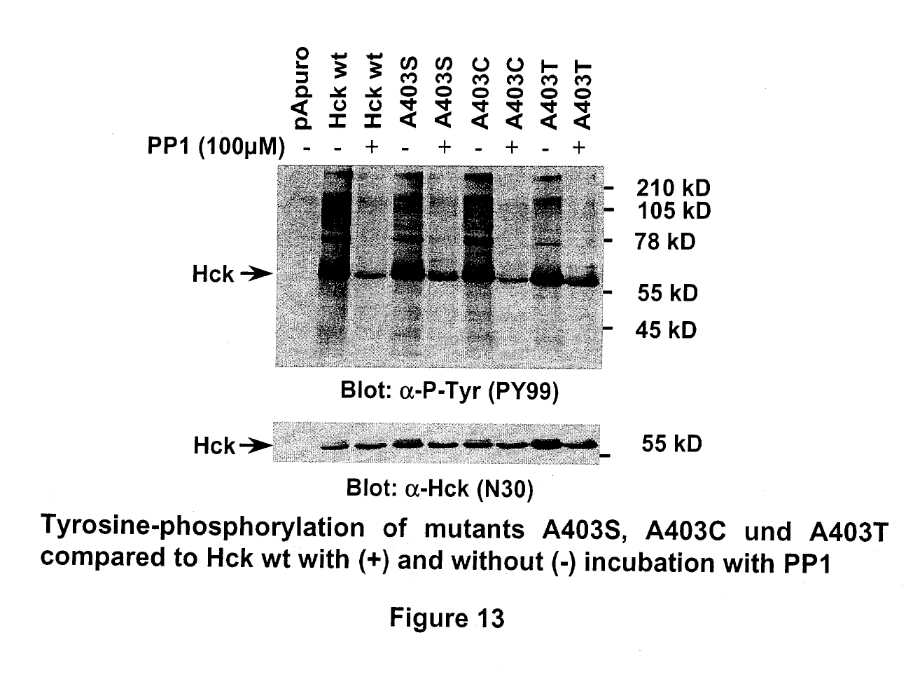

FIG. 13 shows the tyrosine-phosphorylation activity of mutants A403S, A403C and A403T compared to wt Src kinase Hck with (+) and without (−) incubation with PP1.

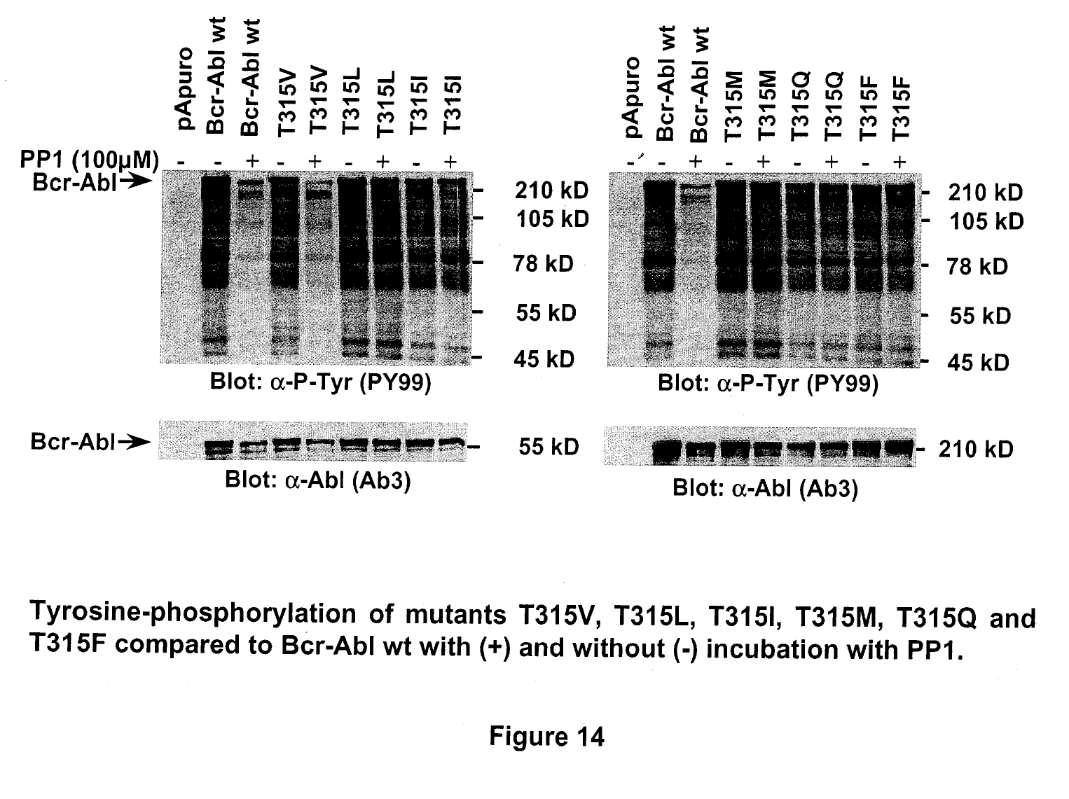

FIG. 14 shows the tyrosine-phosphorylation activity of mutants T315V, T315L, T315I, T315M, T315Q and T315F compared to wt Bcr-Abl with (+) and without (−) incubation with PP1;

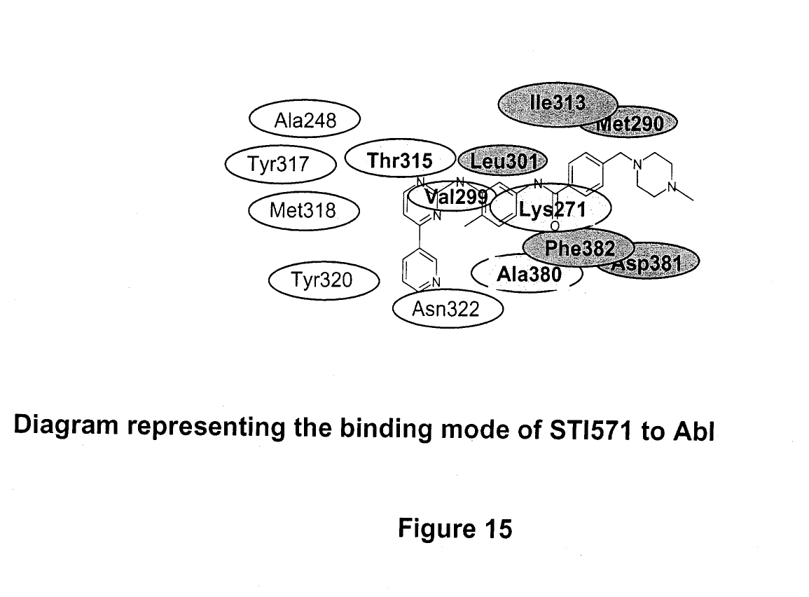

FIG. 15 is a diagram representing the postulated mode of binding STI571 to protein kinase Abl;

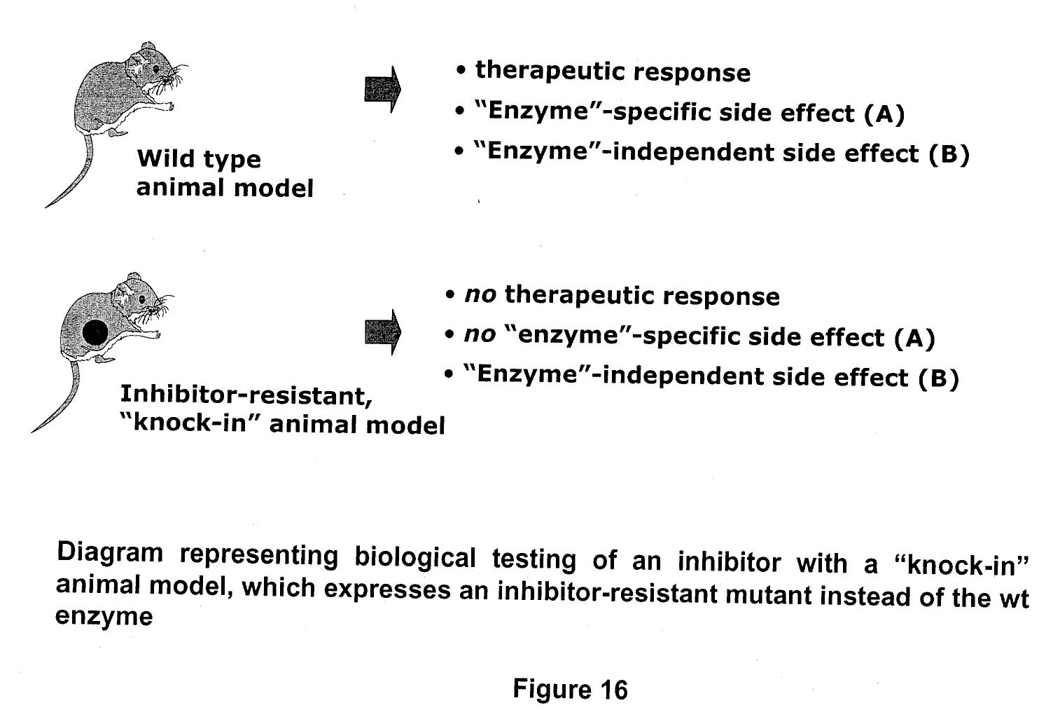

FIG. 16 is a diagram representing biological testing of an inhibitor with a “knock-in” animal model, which expresses the inhibitor-resistant mutant instead of the wt enzyme;

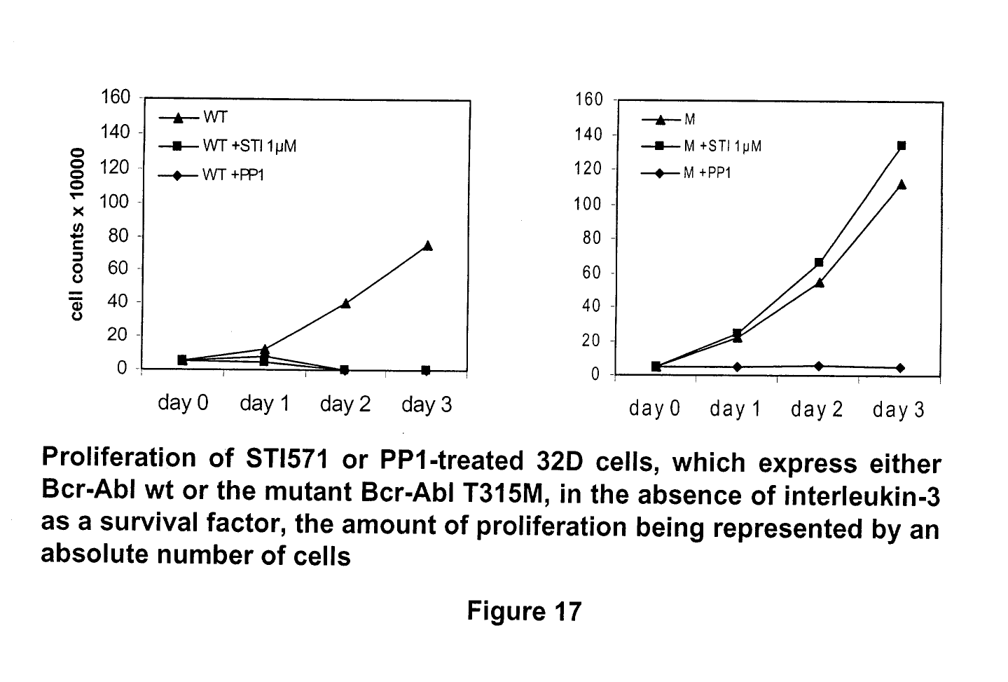

FIG. 17 contains two diagrams showing the proliferation of STI571 or PP1-treated 32D cells, which express either wt Bcr-Abl or the mutant Bcr-Abl T315M, in the absence of interleukin-3 as a survival factor, the amount of proliferation being represented by an absolute number of cells;

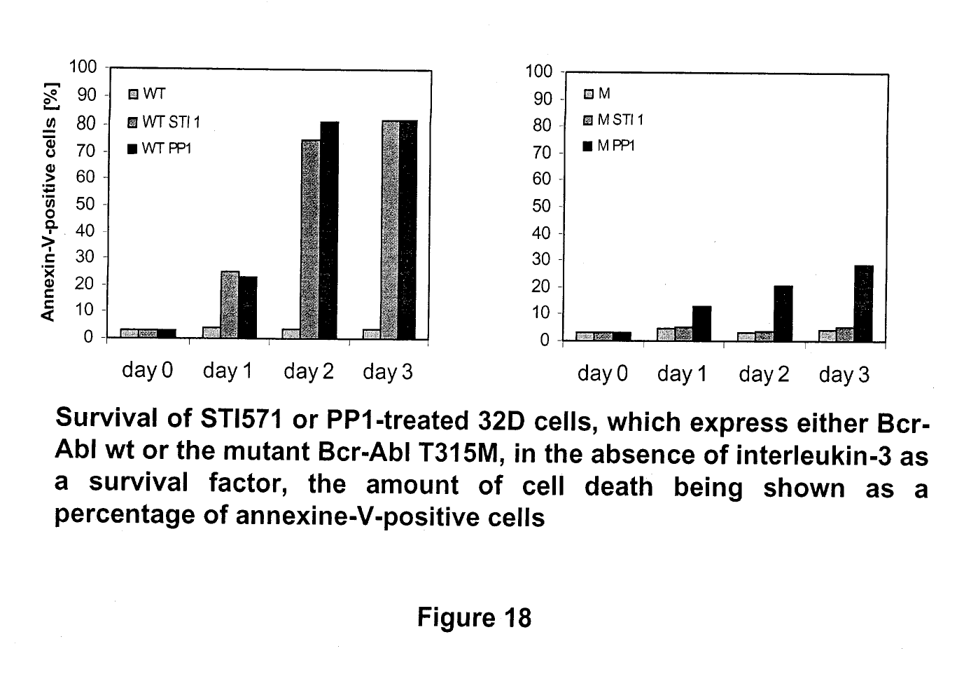

FIG. 18 contains two diagrams showing the survival of STI571-treated or PP1-treated 32D cells, which express either wt Bcr-Abl or the mutant Bcr-Abl T315M, in the absence of interleukin-3 as a survival factor, the amount of cell death being represented by the percentage of annexine-V-positive cells;

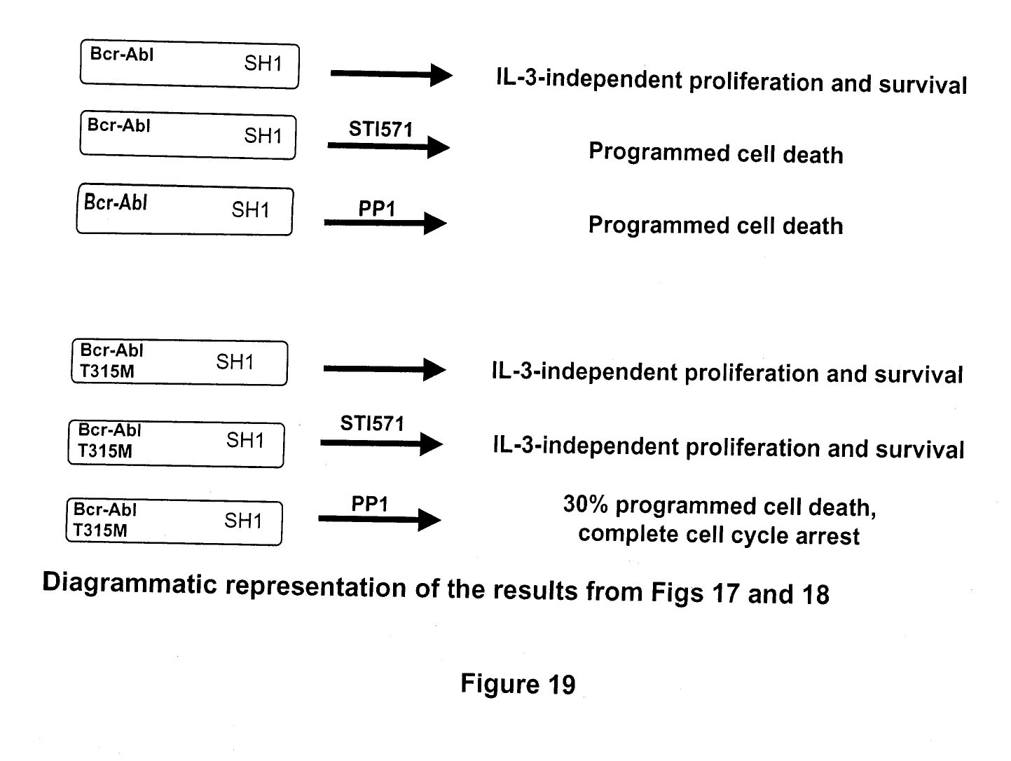

FIG. 19 is a diagram showing the results from FIGS. 17 and 18; and

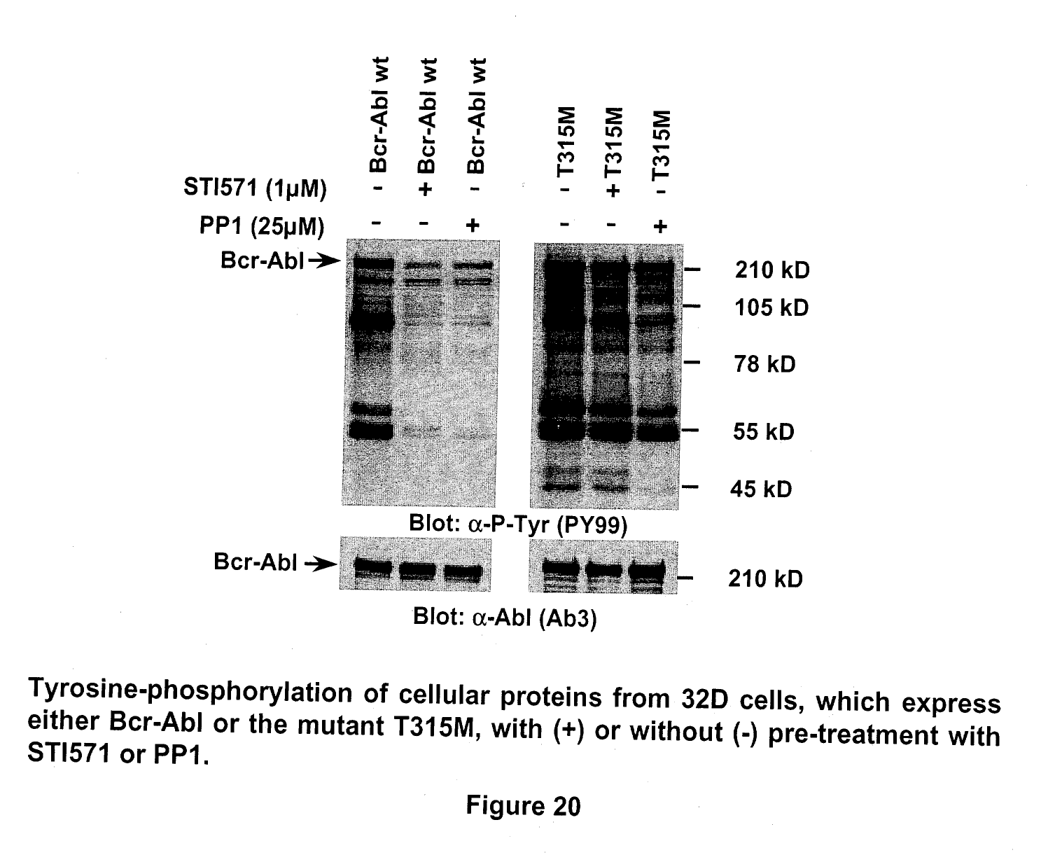

FIG. 20 shows the tyrosine-phosphorylation of cellular proteins from 32D cells, which express either wt Bcr-Abl or the mutant Bcr-Abl T315M, with (+) or without (−) pre-treatment with STI571 or PP1.

According to the invention the method concerns the selection of inhibitors with optimum binding properties to a certain selected enzyme, wherein specific inhibitors are found. At step a) of the method of the invention an inhibitor-specific but not substrate-specific binding site is determined in a wt enzyme. This may be done by analysing the crystal structure of a wt enzyme for its substrate-specific binding site and the steric arrangement of a known inhibitor for the wt enzyme. In this way the binding site specific only to the inhibitor and not to the substrate can be found. An inhibitor-specific but not substrate-specific binding site may alternatively be determined without co-crystallisation of the wt enzyme with a known inhibitor, i.e. merely based on crystallisation of the enzyme with its substrate. Or instead of the crystal structure a tertiary structure of a wt enzyme determined by calculation based on the amino acid sequence may be used. The enzyme used at step a) is preferably selected from the group comprising protein kinases, proteases and phosphatases. The protein kinase is preferably chosen from the group comprising Src kinases Src, Lyn, Fyn, Hck, Lck, Blk, Yes, Yrk, Fgr, kinases ZAP70, BTK, Tec, Jak1, Jak2, PKA, VEGF-family, PDGF-family and EGF-family receptor kinases, MAP kinases, c-Abl and cyclin-dependent kinases, particularly Src kinases Hck or Lyn and kinase c-Abl.

It is particularly preferable to use Src kinase Hck and kinase Abl at step a). In the following description known inhibitor PP1 is used with Src kinase Hck and known inhibitors PP1 and STI571 with tyrosine kinase Abl, thereby demonstrating that inhibitors specific to the enzymes can be found by the method of the invention.

In one embodiment the crystal structures of Src kinase Hck in a complex with PP1 and Src kinase Lck in a complex with PP2, published by Schindler et al. in Molecular Cell, 1999, 3, pp. 638-648 and by Zhu et al. in Structure, 1999, 7, pp. 651-616, were used to determine the structural requirements for the specificity of inhibitors for Src kinases.

Both inhibitors bind to the ATP binding site of the kinase domain and competitively displace the ATP, which is important for phosphorylation of the substrate. The binding mode of the pyrazolopyrimidine skeleton corresponds approximately to that of the purine skeleton of ATP. However the 4-methylphenyl radical of PP1 and the 4-chlorophenyl radical of PP2 also move into a hydrophobic pocket, the entrance to which is bounded by the amino acid side chains of lysine (K) in position 295, valine (V) in position 323, threonine (T) in position 338 and alanine (A) in position 403, as illustrated in FIG. 1. The hydrophobic pocket is therefore an inhibitor-specific but not substrate-specific binding site.

Comparisons of the sequence with other protein kinases—e.g. Flk1, Met, Tie1, Jak1, Erk1 or Flt3, show that these have different amino acids, particularly in positions homologous with T338 and A403, as shown in FIG. 2. For example phenylalanine instead of threonine is found in Flt3 kinase at the position homologous with T338, and cysteine instead of alanine at the position homologous with A403. Comparison of the three-dimensional structures of Hck and a computer-simulated mutant of Hck in position 338 with phenylalanine shows that these differences in the amino acid sequence cause considerable narrowing of the entrance to the above-mentioned hydrophobic pocket. These positions thus act as so-called ,,molecular gatekeepers” or specificity determinants. Compared to them considerable homology is found within the protein kinase family with the positions correlating with lysine 295 and valine 323.

The hydrophobic pocket itself is bounded inter alia by the amino acids methionine in position 314, leucine in position 325, isoleucine in position 336, aspartate in position 404 and phenylalanine in position 405, as also shown in FIG. 1. As described in connection with amino acids T338 and A403, differences in the amino acid sequence similarly affect the space and hydrophobic character conditions inside the hydrophobic pocket and thus the binding of the inhibitors to the binding site.

In another embodiment a potential inhibitor-specific binding site in tyrosine kinase Abl is identified, based on comparisons of sequence homology and a computer model. It is a pocket which is 100% homologous with the hydrophobic pocket of Src kinase Hck described above, as will be seen from FIGS. 4 and 5. The inlet region to the pocket is formed by amino acids lysine271, Val299, Thr315 and Ala380. Amino acid alignments with other tyrosine kinases show that, as already described in connection with Hck, the other tyrosine kinases have different amino acids particularly in positions homologous with T315 and A380, as shown in FIG. 4. These amino acids have a longer side chain, so that entrance to the pocket described above is critically restricted or closed.

It has thus been shown with two examples how the inhibitor-specific but not substrate-specific binding site in a wt enzyme can be determined by means of a known crystal structure or based on comparisons of sequence homology and a computer model.

At step b) of the method of the invention at least one amino acid at the binding site determined as being inhibitor-specific is substituted by a different amino acid. The number of mutated positions and the type of amino acids inserted in the substitution depends on the structure of the binding site found to be inhibitor-specific. As a rule the main amino acid positions changed are those which have maximum variability within an enzyme family, i.e. which are occupied with amino acids as differently as possible within an enzyme family.

At step b) the amino acid is preferably substituted by an amino acid which takes up more space or is more hydrophobic, more hydrophilic, more basic or more acid.

If the binding site found to be inhibitor-specific has to be changed spatially so as to prevent the inhibitor from accessing it, it is preferable to choose replacement amino acids which take up more space than the amino acid replaced. In this context taking up more space may mean that the new amino acid has a longer or more voluminous side chain than the one replaced. It is preferable to carry out a plurality of amino acid substitutions for each position, so that there is a gradual change in spatial conditions in the region of an inhibitor-specific binding site. Mutants of this type are combined into libraries.

It is also possible to adapt the properties of the binding site found to be inhibitor-specific so that the hydrophobic or hydrophilic nature of the site is changed. For example, if the binding site is built up largely with hydrophobic amino acids, its hydrophobic nature can be changed by making the new amino acid a hydrophilic one.

In one embodiment a wide variety of amino acid substitutions are made in Src kinase Hck at positions 338 and 403, as shown in FIG. 5. The numbering of the positions refers to the homologous position in c-Src from the chicken. The new amino acids take up more space than those in wt Src-kinase Hck. As determined in the analysis of crystal structures and shown in FIG. 1, these positions are at the entrance to the hydrophobic pocket which is the inhibitor-specific binding site. As the new amino acids take up more space, the entrance to the hydrophobic pocket becomes smaller. The choice of amino acids inserted by exchange is based on comparisons of sequence homology, i.e. only amino acids which occupy the homologous position in other kinases are inserted. It is preferable to consider kinases from the same species, e.g. from humans.

In one embodiment Src kinase Hck is used at step b), preferably with threonine at position 338 and alanine at position 403 being replaced by a different amino acid.

Threonine at position 338 is preferably replaced by an amino acid chosen from the group comprising valine, leucine, isoleucine, methionine, glutamine and phenylalanine, and alanine at position 403 by an amino acid chosen from the group comprising serine, cysteine and threonine. These are the amino acids found at the homologous position in other kinases. The corresponding mutations lead to a gradual narrowing of the entrance to the hydrophobic pocket, as shown in FIG. 6.

In Src kinase Hck it is possible to replace methionine at position 314, leucine at position 325 and isoleucine at position 336 with phenylalanine and/or threonine. This reduces the hydrophobic character of the hydrophobic pocket on the one hand through the substitution with threonine, and makes the space inside the pocket smaller through the substitution with phenylalanine. There is no point in mutating aspartate at position 404 and phenylalanine at position 405 owing to their important catalytic function.

In another embodiment threonine at position 315 in tyrosine kinase Abl is substituted by amino acids valine, leucine, isoleucine, methionine, glutamine and phenylalanine. Also in Abl, alanine at position 380 is substituted by amino acids serine, cysteine and threonine. These are the amino acids found at the homologous position in other kinases, as shown in FIG. 7. The corresponding mutations lead to a gradual narrowing of the entrance to the potential inhibitor binding site pocket, as shown in FIG. 8.

Again in kinase Abl, methionine at position 290, leucine at position 301 and/or isoleucine at position 313 may be substituted by phenylalanine and/or threonine. In this way the hydrophobic character of the hydrophobic pocket is on the one hand reduced through the substitution with threonine, and the space inside the pocket is made smaller through the substitution with phenylalanine. There is no point in mutating aspartate at position 381 and phenylalanine at position 382, owing to their important catalytic function.

Substitution of the amino acids of the enzyme, e.g. Src kinase Hck or Lck, may be carried out by conventional mutagenesis processes such as PCR mutagenesis. In PCR mutagenesis the gene to be mutated is inserted in a cloning vector. Using DNA primers containing mutagenic codons, the appropriate mutation in the gene is introduced by a standard PCR reaction. The mutated gene is then amplified in bacteria and the mutation confirmed by sequencing.

By carrying out step b) mutants of the wt enzyme are obtained, which are changed sterically or in respect of their hydrophilic or hydrophobic properties or their basicity or acidity compared to the wt enzyme, at the inhibitor-specific binding site.

In step c) of the method the mutants obtained in step b) are thereupon tested as to whether they still show the enzyme activity of the wt enzyme. The mutants where substitution of the amino acids in step b) does not affect enzyme activity, i.e. the active mutants, are then selected.

In one embodiment the tyrosine-phosphorylation activity of mutants of Src kinase Hck is tested as shown in FIG. 9. In the mutants used threonine at position 338 is substituted by valine, leucine, isoleucine, methionine, glutamine and phenylalanine, and alanine at position 403 by serine, leucine and threonine. The respective mutants are expressed in Cos7 cells.

These cells are lysised and the protein extracts thus obtained are thereupon examined for cellular tyrosine phosphorylation of the substrate by Western Blotting. This is done using an antibody (PY99) which specifically recognises tyrosine-phosphorylated proteins: The result of this assay for the various mutations at positions 338 and 403 is shown in FIG. 9. It will be seen from FIG. 9 that expression of wt Hck in Cos7 cells leads to the induction of many phosphorylation actions. Three mutations, T338L, T338I and T338M, lead to a clear increase in the phosphorylation of cellular proteins induced by Hck. These mutants are accordingly more active than wt Hck. Five other mutants, T338V, T338Q, T338F, A403S and A403C, show approximately the same activity as wt Hck. One mutant, A403T, shows slightly less activity than wt Hck.

It will thus be seen from FIG. 9 that substitution of threonine at position 338 by leucine, isoleucine and methionine leads to hyperactivation, and substitution by valine, glutamine and phenylalanine leads to activity comparable with wt Src kinase Hck. Exchange of alanine at position 403 for serine and cysteine also gives activity comparable with wt Src kinase Hck. Exchange for threonine produces a slight reduction in kinase activity.

In a further embodiment the tyrosine-phosphorylation activity of mutants of the kinase Abl and the leukaemia-inducing sub-form of Abl, Bcr-Abl, is tested as shown in FIG. 10. In the mutants used threonine at position 315 is in each case exchanged for valine, leucine, isoleucine, methionine, glutamine and phenylalanine. The respective mutants are expressed in Cos7 cells.

These cells are lysised and the protein extracts thus obtained are then examined for cellular tyrosine phosphorylation of the substrate by Western Blotting. This is done as described above, using antibody PY99, which specifically recognises tyrosine-phosphorylated proteins. The result of the assay for the various mutations at position 315 is shown in FIG. 10. It will be seen from FIG. 10 that expression of wt Bcr-Abl in Cos7 cells leads to the induction of many phosphorylation reactions. All the mutants of kinase Abl at position 315 produced in this embodiment show approximately the same tyrosine-phosphorylation activity as wt Bcr-Abl, in respect of the phosphorylation of cellular substrates by the expressed mutants.

It will thus be seen from FIG. 10 that none of the amino acid substitutions made at position 315 substantially change the tyrosine-phosphorylation properties of Bcr-Abl.

At step c) all the mutants with an enzymatic action can be selected for carrying out steps d) and e) of the method of the invention. It is preferable as far as possible to select a plurality of mutants with the most gradual possible gradation of the changes to the inhibitor-specific binding site. If a mutant no longer has any enzyme action, the change of an amino acid at the inhibitor-specific binding site can be assumed to have affected the tertiary structure of the enzyme so much that the mutant is no longer suitable for use in the following steps to select a specific inhibitor for the wt enzyme. The mutant selected in step c) thus has maximum structural similarity with the wt enzyme, except that the most gradual possible change has been made at the inhibitor-specific binding site of the enzyme.

In one embodiment mutants T338V, T338L, T338I, T338M, T338Q, T338F, A403S, A403C and A403T are selected for Src kinase Hck. These mutants all have sufficient kinase activity to be used for steps d) and c).

In another embodiment mutants T315V,T315L, T315I, T315M, T315Q and T315F are selected for kinase Abl. These mutants all have sufficient kinase activity to be used for steps d) and c).

If a specific inhibitor for an enzyme is already known, as is the case in these embodiments, then in a preferred embodiment the mutants selected in step c) of the method of the invention may additionally be tested to establish whether they are inhibited by the known inhibitor. The mutants, which are no longer inhibited by the known inhibitor, are then selected.

In step d) of the method of the invention at least one substance is tested with the wt enzyme and the mutants selected in step c).

A substance library consisting of a wide variety of substances is preferably tested simultaneously. This makes it possible to distinguish between 3 different categories of substance, namely (1) substances which do not inhibit either the wt enzyme or the mutant selected in step c) (not inhibitors), (2) substances which inhibit both the wt enzyme and the mutant selected in step c) (unspecific inhibitors), and (3) substances which inhibit only the wt enzyme and not the mutant selected in step c), as illustrated diagrammatically in FIG. 11. The substances (3) which inhibit only the wt enzyme and not the mutant selected in step c) are then selected as inhibitors.

A substance or substance library is preferably tested with a plurality of mutants selected in step c), preferably with at least 2, in particular with at least 5 and particularly preferably with at least 10 mutants. In this embodiment the substance which inhibits only the wt enzyme and none of the selected mutants is preferably selected.

In one embodiment the question of whether the known Src inhibitor PP1 can be selected by the method of the invention is considered. For this purpose the tyrosine-phosphorylating activity of the mutants obtained by substitution of threonine at position 338 by valine, leucine, isoleucine, methionine, glutamine and phenylalanine and by substitution of alanine at position 403 by serine, cysteine and threonine in wt Src kinase Hck is tested with and without adding inhibitor PP1, as shown in FIGS. 12 and 13. It will be seen from FIGS. 12 and 13 that the threonine to leucine, isoleucine, methionine, glutamine and phenylalanine mutants are not inhibited by PP1. The threonine 315 to valine mutants and alanine 403 to serine, cysteine and threonine mutants are inhibited by PP1, like the wt form of Hck. PP1 is thereby selected as a substance which binds into the hydrophobic pocket, i.e. the inhibitor-specific binding site. For this purpose the mutants are expressed in Cos7 cells as described above in connection with step c), except that they are additionally incubated with PP1 prior to lysis.

Expression of wt Hck causes a marked increase in the tyrosine phosphorylation of many cellular proteins. Incubation of the cells with 100 μM PP1 prior to lysis cancels the phosphorylation induced by wt Hck or considerably reduces it. The same applies to mutants T338V, A403S, A403C and A 403T. In contrast with this the phosphorylation induced by mutants T338L, T338I, T338M, T338Q and T338F remains largely unchanged even after incubation with PP1. Thus these mutants are resistant to inhibition by PP1. Hence adequate narrowing of the entrance to the hydrophobic PP1-binding pocket by lengthening the amino acid side chain at position 338 induces resistance to PP1 without losing the basic activity of the enzyme. This result confirms the importance of the hydrophobic PP1-binding pocket to the binding of PP1. Conversely narrowing of the entrance to the hydrophobic ,,inhibitor-binding pocket” by lengthening the amino acid side chain at position 403 does not create resistance to PP1.

Table 1 below summarises examples of amino acid substitutions made in kinase Hck and the results of the tests carried out in steps c) and d).

TABLE 1

| Mutants | Kinase activity | Inhibited by PP1 |
|---|---|---|
| wt | +++ | yes |
| Thr338Val | ++++ | yes |
| Thr338Leu | +++++ | no |
| Thr338Ile | ++++ | no |
| Thr338Met | ++++ | no |
| Thr338Gln | +++ | no |
| Thr338Phe | +++ | no |
| Ala403Ser | +++ | yes |
| Ala403Cys | +++ | yes |
| Ala403Thr | −++ | yes |

As shown in the Table, five of the mutants, namely T338L T338I, T338M, T338Q and T338F, although still having kinase activity, could no longer be inhibited by PP1. Thus PP1 would be chosen as an inhibitor in carrying out the method of the invention.

In a further embodiment the tyrosine-phosphorylating activity of tyrosine kinase Abl or its leukaemia-inducing form, Bcr-Abl, and the mutants of Bcr-Abl at position 315 to valine, leucine, isoleucine, methionine, glutamine and phenylalanine are tested with and without adding known kinase inhibitors. The inhibitors used are PP1 and STI571. The aim is to determine which inhibitors bind into the potential ,,inhibitor-binding pocket” of Abl kinase and thus have the greatest possible specificity to other protein kinases.

For this purpose the mutants are expressed in Cos7 cells, as described above in connection with tyrosine kinase Hck, except that they are additionally incubated with PP1 or STI571 prior to lysis.

FIG. 14 representatively shows the results for incubation of Bcr-Abl wt and the mutants of Bcr-Abl at position 315 to valine, leucine, isoleucine, methionine, glutamine and phenylalanine with PP1. Expression of Bcr-Abl wt causes a marked increase in the tyrosine phosphorylation of many cellular proteins. Incubation of cells with inhibitor PP1 prior to lysis cancels the phosphorylation induced by Bcr-Abl wt or considerably reduces it. Tyrosine phosphorylation induced by T315V mutant is also cancelled. On the other hand tyrosine phosphorylation induced by mutants T315L, T315I, T315M, T315Q and T315F is not cancelled by PP1. These mutants are thus resistant to PP1. This confirms that PP1 can bind as an inhibitor into the hydrophobic ,,inhibitor-binding pocket” of Bcr-Abl.

Table 2 below gives the results of inhibiting Bcr-Abl and mutants of Bcr-Abl at positions 315 and 380 with PP1 and STI571.

TABLE 2

| | Kinase | Inhibition by | Inhibition by |
|---|---|---|---|
| Mutant of tyrosine kinase Abl | activity | PP1 | ST1571 |
| Bcr-Abl wt | +++ | + | + |
| Thr 315 Val | +++ | + | − |
| Thr 315 Ile | +++ | − | − |
| Thr 315 Leu | +++ | − | − |
| Thr 315 Met | +++ | − | − |
| Thr 315 Gln | +++ | − | − |
| Thr 315 Phe | +++ | − | − |
| Ala 380 Ser | +++ | + | + |
| Ala 380 Cys | +++ | + | + |
| Ala 380 Thr | +++ | + | − |

As shown by the results in Table 2, PP1 still inhibits mutants T315V, A380S, A380C and A380T. Mutants T315L, T315I, T315M, T315Q and T315F can no longer be inhibited by PP1. Compared with these results STI571 only inhibits mutants A380S and A380C. Mutants T315V, T315L, T315I, T315M, T315Q and T315F and A380T can no longer be inhibited. Given adequate elongation of the amino acid side chains at positions 315 and 380 therefore, entry to the hydrophobic inhibitor-binding pocket is barred for STI571. This shows that STI571, like PP1, binds into the hydrophobic ,,inhibitor binding pocket” of Bcr-Abl, and that STI571 fills the pocket better than PP1, based on the gradual gradation of the mutants. Inhibitor STI571, which is known to be very specific to tyrosine kinase Abl and is involved in clinical trials, is therefore selected by the method of the invention in comparison with PP1.

In step e) of the method the substance which inhibits the wt enzyme but not the mutants selected in step c), as shown in FIG. 11, is selected as inhibitor. The substances preferably selected are those which inhibit the wt enzyme but not those mutants with the smallest structural change, for example with only slight elongation of the amino acid side chain at a position identified as a specificity determinant. Following the method of the invention, in the example described above, PP1 is selected as inhibitor for Src kinase Hck and STI571 as inhibitor for Abl. in step e). STI571 is therefore selected as the inhibitor for Abl rather than PP1 because, unlike PP1, it can no longer inhibit mutants T315V and A380T. Based on the gradual changes made in Abl by the respective mutations at positions 315 and 380 (cf. FIG. 8), STI571 fills the inlet to the hydrophobic binding pocket better.

The method described above simplifies selective screening for lead structures which can be considered as specific inhibitors for enzymes such as Src kinases or Abl kinase. By using mutants where essential specificity determinants for interaction between an inhibitor and the inhibitor-specific binding site has been eliminated or changed in gradual stages, the screening process is considerably simplified, as screening is now carried out for the inhibitor-resistant mutant and not for a number of other kinases which are potentially cross-inhibitable. Development periods and expenses for such a test set-up are altogether considerably less. In addition selection for structural features which give potential inhibitors the highest possible specificity compared with other potential target molecules can take place even at the lead identification stage.

Apart from high-throughput screening, structure based, molecular drug design is becoming increasingly important. It is therefore conceivable, based on the crystal structure of Src kinases on the one hand and already known lead structures for tyrosine kinase inhibitors on the other that modified and optimised inhibitors could be computer modelled. With regard to Src kinases it would be helpful to model substances which utilise the hydrophobic PP1-binding pocket as far as possible optimally for binding. With the mutants thus produced one can then consider to what extent prediction agrees with reality, i.e. whether or not a computer-modelled and optimised inhibitor does in fact use the PP1-binding pocket for interaction with the target.

As will be seen from the two above examples dealing with Src kinase Hck and kinase Abl, known inhibitors for these two kinases are recognised successfully by the method of the invention. In particular, comparison of the method of the invention in the screening of PP1 and STI571 for mutants and the wt enzyme Abl shows that the more specific of the two inhibitors, STI571, is selected by the method of the invention.

The method of the invention can find inhibitors which are highly specific to inhibition of certain enzymes. They may be applied in many different fields. In particular they may be used as therapeutic and/or prophylactic agents for treating diseases such as cancerous conditions, allergies, transplant-rejecting reactions and/or osteoporosis. The agent according to the invention can preferably treat cancerous conditions such as leukaemias or solid tumours.

In a further embodiment of the invention a method is provided for testing an inhibitor, which has been selected by the method of the invention, for biological effects specific to interaction between the inhibitor and the inhibitor-specific binding site, comprising the steps of:

1) incubating a modelling system, in which the wt enzyme is expressed, with an inhibitor,

2) establishing the effects thereby obtained,

3) incubating a modelling system, in which a mutant according to claim 12 is expressed, with the inhibitor,

4) establishing the effects thereby obtained,

5) comparing the effects found at step 2) and step 4) and

6) selecting the effects found at step 2) but not step 4), as being specific to the interaction between the inhibitor and the inhibitor-specific binding site.

The modelling system used in the method of the invention is preferably selected from the group comprising cell lines, microorganisms and animals. The animals used may be mice, rats or rabbits. The cell lines used are preferably cell lines 32D and BaF3, which are both modelling systems for leukaemia.

The effects established at step 2) are preferably selected from the group comprising therapeutic effects, acute and sub-acute organ toxicity, non-therapeutic immuno-suppression and lethal effects.

The above-mentioned, inhibitor-resistant mutants may thus be employed for further biological validation of selected and optimised inhibitors or inhibitor lead structures. Enzyme-specific effects of the inhibitor can be distinguished by the method of the invention, and thus desired effects e.g. therapeutic effects can be distinguished from enzyme-independent effects of the inhibitor, i.e. undesirable effects.

As shown in FIG. 16 these mutants can differentiate between enzyme-specific and enzyme-independent effects of the inhibitor, particularly between enzyme-specific and enzyme-independent side effects. In the top part of FIG. 16 incubation of a modelling system such as a mouse with an inhibitor produces firstly a therapeutic effect and secondly (side) effects A and B. The modelling system may alternatively be a cell line. As shown in the bottom part of FIG. 16, an inhibitor-resistant allele may be expressed in a ,,knock-in” mouse in this modelling system. The corresponding gene product (IR) can no longer be inhibited by the inhibitor and can thus fulfil its function even when the inhibitor is present. This test batch cancels enzyme-specific effects of the inhibitor. As effect B still occurs it must be an enzyme-independent effect.

To produce a ,,knock-in” mouse which can be used in the invention, embryonic stem cells (ES cells) are cultivated. A transfer vector is inserted in these cells, the vector containing not only the gene of interest but also the regions flanking it in the genome and a selection marker, i.e. a resistance gene. The wt gene is substituted by the “knock-in” gene by homologous recombination. The ES cells in which the desired gene exchange took place are then selected by means of the resistance marker. The ES cells thus selected are injected into mouse blastocysts, and these are implanted in spuriously pregnant mice. Chimeric offspring are thus obtained and are eventually used to produce genetically pure offspring by crossing.

By using this test batch (knock-in strategy) even before clinical studies are commenced it is possible to differentiate between inhibitor-specific and inhibitor-independent effects. This is of considerable value in predicting any side effects of use of the inhibitor. The method of the invention provides important help in reaching decisions during the development of an inhibitor. If for example a plurality of enzyme-independent, undesirable side effects occur when an inhibitor is tested in the wild type or the inhibitor-resistant mouse, this suggests additional interaction between the inhibitor and a further target structure inside the organism, which is different from the therapeutic target structure. Accordingly either the specificity of the inhibitor has to be increased by appropriate modifications or a different substance has to be found, which does not produce undesirable, enzyme-independent side effects. If on the other hand a plurality of enzyme-dependent side effects occur, the enzyme is called into question as being a therapeutically disputable target structure, and a better, therapeutically helpful enzyme accordingly has to be sought for the appropriate disease. This process of decision and development may save high expenditure on subsequent unsuccessful clinical studies.

The enzyme or target structure specificity of the therapeutic response is also clarified by the method of the invention. Knowing about the enzyme or target structure specificity of the therapeutic response may in turn considerably speed up the registration process for a medicine containing the inhibitor.

Not only animal models but also cell culture models may be used for biological validation of inhibitors by means of the method of the invention.

In one example of the method of the invention either Bcr-Abl or a mutant previously identified as being resistant to kinase inhibitors PP1 and STI571, e.g. T315M, is expressed in a murine, interleukin-3-dependent cell line, 32D. Bcr-Abl is a constitutionally active tyrosine kinase which induces different forms of leukaemia in the animal model and humans.

The example is carried out by transfixing 32D cells with a plasmid by electroporation, i.e. with the aid of a current pulse. The plasmid codes for either wt Bcr-Abl or e.g. for the mutant T315M. These plasmids additionally carry a resistance marker; in this case a puromycin resistance gene. Transfixed cells are therefore selected by means of puromycin. After their selection some of the cells are lysised and the expression of Bcr-Abl is demonstrated by Western blot analysis.

The cells are then characterised biologically. For this purpose interleukin-3 is removed from them as an essential survival and growth factor. Whereas untransfixed 32D cells cease proliferating and die within 24 hours when interleukin-3 is removed, both cells which have been transfixed with wt Bcr-Abl and cells which have been transfixed with a mutant of Bcr-Abl at position 315, e.g. T315M, can survive and proliferate even without the presence of interleukin-3 in the culture medium (FIGS. 17 and 18), and can do so to the same extent. This shows that the mutation of Bcr-Abl at position 315, e.g. to methionine, does not cancel or reduce the leukaemogenic power of the kinase (FIG. 17).

In addition either PP1 (25 μM) or STI571 (1 μM) is put into some of the cells. Both substances prevent both proliferation and survival of cells which express Bcr-Abl wt, and do so to the same extent (FIGS. 17 and 18).

The cells which express a mutant of Bcr-Abl at position 315, e.g. T315M, and have been incubated with STI571 show proliferation and survival like untreated cells (FIGS. 17 and 18). STI571 cannot inhibit Bcr-Abl in these cells because the entrance to the inhibitor-specific binding site is blocked by the elongation of the amino acid side chain at position 315. The fact that STI571 has no biological effect in these cells thus proves that Bcr-Abl is the only target molecule of STI571 with a relevant action under these conditions. The action of STI571 is thus ,,target molecule” specific, which suggests that the substance has low toxicity.

In contrast with this result incubation of cells which express mutant T315M with PP1 leads to complete cessation of cell growth and a reduced cell survival rate (FIGS. 17 and 18). Altogether about 30% of the cells die off. As the mutant T315M used is biochemically resistant to PP1 (see above), the result shows that PP1 recognises not only Bcr-Abl in Bcr-Abl-greater toxicity in vivo than STI571. positive 32D cells but also another biologically relevant target structure which causes unspecific toxicity. Hence it can be taken that PP1 has comparatively higher toxicity than STI571.

At step 6 of the method of the invention STI571 is accordingly selected here as the Abl-spezific inhibitor, because its biological effect on Bcr-Abl-expressing 32D cells is purely ,,target molecule” specific. Although PP1 also inhibits Bcr-Abl and leads to cell death, the effect is not absolutely ,,target molecule” specific, so greater toxicity of the inhibitor would be expected in vivo. In fact STI571 is at present being tested in clinical studies, and there have so far been no signs of a relevant toxicity.

To find out whether the biologically relevant target molecule for PP1 in 32D cells which is different from Bcr-Abl is a tyrosine kinase, cells which either express wt Bcr-Abl or the mutant T315M are left untreated or treated with STI571 (1 μM) or PP1 (25 μM) then lysised. The protein extracts thus obtained are then examined for tyrosine phosphorylation of cellular proteins. This is done using the antibody PY99, which specifically recognises tyrosine-phosphorylated proteins. Both Bcr-Abl wt and the mutant T315M are found to induce phosphorylation of many cellular proteins. Incubation of cells which express Bcr-Abl wt with either STI571 or PP1 leads to substantially uniform reduction in tyrosine phosphorylation of Bcr-Abl and other cellular proteins. Incubation of mutant T315M with STI571 has no effect on phosphorylation of cellular proteins, i.e. there is complete resistance to STI571. If cells which express mutant T315M are however incubated with PP1, there is a clear reduction in the phosphorylation of some cellular proteins. This indicates that PP1 is acting on another, biologically relevant tyrosine kinase in these cells. It might for example be kinases of the Src family, which are also inhibited by PP1 (see above). It is also known from the literature that STI571 does not inhibit Src kinases.

The method of the invention may accordingly also be used for detailed molecular analysis of enzyme-unspecific effects.

A process for carrying out the method of the invention is described in detail below.

Cell Cultivation

Cos7 cells are cultivated in Dulbecco's modified Eagle's Medium (DMEM) enriched with 10% fetal calf serum (FCS).

Plasmids and Preparation of Point Mutants

Point mutants are made by cloning the cDNA of the human Hck gene in vector pUC18. This plasmid is then used as an ingredient for a mutagenic polymerase chain reaction (PCR). The PCR is carried out using mutagenic primers which are phosphorylated at their 5′ ends and constructed so that they bind to immediately adjacent regions. The complete plasmid is thus amplified with the standard settings (annealing temperature corresponding to the primer) and with Pfu (Promega) being used as the polymerase. The linear PCR amplificate is then purified, religated to a plasmid by means of T4 DNA ligase and propagated clonally in bacteria. The mutants thus prepared are finally examined for the correctness of the mutation.

For expression in Cos7 cells the mutated Hck alleles are cloned in the EcoRI interface of pApuro vector. This vector is derived from pBabepuro but has a chick actin promoter.

Transfection of Cos7 Cells with Effecten

For transfection of the Hck-mutants in Cos7 cells the cells are sown out fresh 18-24 hours before transfection commences, so that a cell density of 50-75% is reached as transfection begins. Transfection itself is carried out with the aid of transfection reagent Effecten (Qiagen). This is done by taking up 1 μg DNA in 150 μl of PB buffer and mixing for a short time. 8 μl of enhancer is then pipetted in. The batch is mixed again and incubated at room temperature for 2 minutes. 10 μl of transfection reagent Effecten is thereupon added, and the batch is mixed and incubated at room temperature for 10 minutes. The complete batch is taken up in DMEM/10%FCS and carefully pipetted onto the cells to be transfixed. The cells are shaken carefully for a short time, put in a CO₂ incubator and incubated for 48 hours altogether, with the medium being changed 24 hours from the beginning of transfection. 48 hours from the beginning of transfection the cells are lysised.

Treatment of Cos7 Cells with PP1

To study the effect of PP1 on the activity of wild type Hck and the various mutants in vivo, the medium is removed from suitably transfixed cells 4 hours before the beginning of cell lysis and replaced by DMEM/10%FCS containing 25 μM PP1 in dimethyl-sulphoxide (DMSO). Cells are incubated with an equivalent quantity of DMSO without PP1, as a control.

PP1 is obtained from Alexis and stored for a maximum of 4 weeks at 4° C. as 25 mM stock solution.

Lysis of Cos7 Cells

Lysis is effected by removing the medium and dissolving the Cos7 cells with 3 ml trypsin-EDTA solution from the bottom of the culture flask. The cells are then transferred to a 50 ml centrifuging tube with DMEM/10% FCS and centrifuged off.

Lysis of the Cos7 cells is effected by dissolving the cell pellet in 250 μl of lysis buffer (1% NP-40, 20 mM Tris (pH 8.0), 50 mM NaCl, and 10 mM EDTA, 1 mM PMSF, 10 μg/ml aprotinine, 10 μg/ml leupeptine and 2 mM sodium orthovanadate) and transferring it to a 1.5 ml Eppendorf reaction vessel. The batch is mixed briefly with a vortex and incubated for 30 minutes at 4° C. on an overhead rotor. The cell lysate is thereupon centrifuged off for 15 minutes at 4° C. and 14000 rpm. Lastly the excess containing the cytoplasmic proteins is transferred to a new reaction vessel.

SDS-Page and Western-Blotting

To separate the proteins of the cell lysates obtained above, 80 μg of the lysate is mixed 1:1 with 2× specimen buffer and denatured for 5 minutes at 100° C. by SDS polyacrylamide electrophoresis. The specimens are charged fully into the pockets of the gel. The proteins are separated in the electric field and transferred to a nitro-cellulose membrane. Next the membrane is incubated for at least 1 hour in a plastic bowl with 15 ml TBS containing 5% skimmed milk powder, after which it is rinsed 2-3 times with TBS. The membrane is then incubated for 2-18 hours with the primary antibody (anti-phosphotyrosine PY99 from Santa Cruz Biotech, anti-Hck N-30 from Santa Cruz Biotech. in respective dilutions of 1:1000). The antibodies are diluted in 15 ml TBS/1% skimmed milk powder, whereupon the membrane is washed with TBS for 5 minutes three times. The membrane is finally incubated for 1 hour with secondary antibody (donkey-anti-mouse or donkey-anti-rabbit-antibody coupled with horseradish peroxide in a dilution of 1:2000; both antibodies from Amersham), then again rinsed three times for 5 minutes with TBS.

To detect the antibody-marked proteins 1 ml respectively of ECLTM-detection reagents 1 and 2 (Amersham) are mixed and poured onto the membrane in a plastic bowl in the dark room. After exactly 1 minute the detection solution is poured away and the membrane is taken out, drained well and covered with Saran packing film without any bubbles. Finally ECLTM hyperfilms are placed on it for 3-60 seconds and developed in a suitable developing apparatus (Agfa).

1. A method of selecting inhibitors, comprising the steps of
a) determining a binding site in a wt enzyme which is inhibitor-specific but not substrate-specific,
b) replacing at least one amino acid at the binding site of the wt enzyme which is found to be inhibitor-specific with a different amino acid, whereby a mutant of the wt enzyme is obtained,
c) testing the mutants obtained at step b) for enzyme activity and selecting the active mutants
d) testing at least one substance with the wt enzyme and the mutant selected at step c),
e) selecting the substance as inhibitor if it inhibits the wt enzyme but not the mutant selected at step c).

2. The method of claim 1, wherein additionally in step c) the mutant is tested as to whether it can be inhibited by a known inhibitor, and the mutant which is inhibitor-resistant is selected.

3. The method of claim 1 or 2, wherein the enzyme is selected from the group comprising protein kinases, proteases and phosphatases.

4. The method of any of the preceding claims, wherein the protein kinase is selected from the group comprising the Src kinases Src, Lyn, Fyn, Hck, Lck, Blk, Yes, Yrk, Fgr, the kinases ZAP70, BTK, Tec, Jak1, Jak2, PKA, VEGF-family, PDGF-family and EGF-family receptor kinases, MAP kinases, c-Abl and cyclin-dependent kinases.

5. The method of any of the preceding claims, wherein the protein kinase is an Src kinase.

6. The method of claim 5, wherein the Src kinase is Src kinase Hck.

7. The method of any of claims 1 to 4, wherein the protein kinase is tyrosine kinase Abl.

8. The method of any of the preceding claims, wherein in step b) the amino acid is substituted by an amino acid which takes up more space or is more hydrophobic, more hydrophilic, more basic or more acid.

9. The method of claim 5 or 6, wherein at step b) threonine at position 338, alanine at position 403, leucine at position 325, methionine at position 314, and/or isoleucine at position 336 is substituted by a different amino acid.

10. The method of claim 9, wherein threonine at position 338 and alanine at position 403 are substituted by a different amino acid.

11. The method of claim 10, wherein threonine at position 338 is substituted by an amino acid selected from the group comprising valine, leucine, isoleucine, methionine, glutamine and phenylalanine, and/or alanine at position 403 is substituted by an amino acid selected from the group comprising serine, cysteine and threonine.

12. The method of claim 9, wherein methionine at position 314, leucine at position 325 and/or isoleucine at position 336 is substituted by phenylalanine and/or threonine.

13. The method of claim 7, wherein threonine at position 315 is substituted by an amino acid selected from the group comprising valine, leucine, isoleucine, methionine, glutamine and phenylalanine and/or alanine at position 380 is substituted by an amino acid selected from the group comprising serine, cysteine and threonine.

14. The method of claim 7, wherein methionine at position 290, leucine at position 301 and/or isoleucine at position 313 is substituted by phenylalanine and/or threonine.

15. The method of any of the preceding claims, wherein a plurality of substances are tested simultaneously at step e).

16. A mutant of a wt enzyme obtainable by carrying out steps a) and b) of the method of any of the preceding claims.

17. A mutant of Src kinases Hck or Lyn, wherein threonine at position 338, alanine at position 403, methionine at position 314, leucine at position 325 and/or isoleucine at position 336 is substituted by a different amino acid.

18. A mutant according to claim 17, wherein threonine at position 338 and alanine at position 403 are substituted by a different amino acid.

19. A mutant according to claim 18, wherein threonine at position 338 is substituted by an amino acid selected from the group comprising valine, leucine, isoleucine, methionine, glutamine and phenylalanine, and/or alanine at position 403 is substituted by an amino acid selected from the group comprising serine, cysteine and threonine.

20. A mutant according to claim 19, wherein threonine at position 338 is substituted by an amino acid selected from the group comprising valine, leucine, isoleucine, methionine, glutamine and phenylalanine.

21. A mutant according to any of claims 17 to 20, wherein methionine at position 314, leucine at position 325 and/or isoleucine at position 336 is substituted by phenylalanine and/or threonine.

22. A mutant of tyrosine kinase Abl, wherein threonine at position 315 is substituted by an amino acid selected from the group comprising valine, leucine, isoleucine, methionine, glutamine and phenylalanine and/or alanine at position 380 is substituted by an amino acid selected from the group comprising serine, cysteine and threonine.

23. A mutant of tyrosine kinase Abl, wherein methionine at position 290, leucine at position 301 and/or isoleucine at position 313 is substituted by phenylalanine and/or threonine.

24. Use of a mutant according to any of claims 16 to 23 in carrying out the method of any of claims 1 to 15.

25. An inhibitor selected as a prophylactic and/or therapeutic agent by the method of any of claims 1 to 15.

26. Use of an inhibitor selected by the method of any of claims 1 to 15 for preparing a prophylactic and/or therapeutic agent for treating cancers, allergies, rejection reactions with transplants and/or osteoporosis.

27. Use according to claim 26, wherein the cancers are leukaemias or solid tumours.

28. A method of testing an inhibitor selected by a method according to any of claims 1 to 15, for biological effects specific to the interaction between the inhibitor and the inhibitor-specific binding site, comprising the steps of
1) incubating a modelling system in which the wt enzyme is expressed, with an inhibitor,
2) establishing the effects thereby obtained,
3) incubating a modelling system, in which a mutant according to any of claims 16 to 23 is expressed, with the inhibitor,
4) establishing the effects thereby obtained,
5) comparing the effects found at step 2) and step 4), and
6) selecting the effects found at step 2) but not step 4), as being specific to the interaction between the inhibitor and the inhibitor-specific binding site.

29. The method of claim 28, wherein the modelling system is a cell line, microorganism or animal.

30. The method of claim 29, wherein the animal is selected from the group comprising mice, rats and rabbits.

31. The method of any of claims 28 to 30, wherein the effects are selected from the group comprising therapeutic effects, organ toxicity, non-therapeutic immuno-suppression and lethal effects.
